(19) United States
(12) Patent Application Publication
(10) Pub. No.: US 2010/0298567 A1
(43) Pub. Date: Nov. 25, 2010

(54) NITRATION OF ACTIVATED AROMATICS IN MICROREACTORS

(75) Inventors: Dominique Roberge, Sierre (CH); Laurent Ducry, Sierre (CH)

Correspondence Address: HOFFMANN & BARON, LLP, 6900 JERICHO TURNPIKE, SYOSSET, NY, 11791, US

(73) Assignee: LONZA AG, BASEL (CH)

(21) Appl. No.: 12/160,804

(22) PCT Filed: Jan. 12, 2006
(86) PCT No.: PCT/EP06/00241

§ 371 Date: Aug. 10, 2010

(51) Int. Cl. C07D 239/02; C07C 205/00; C07C 63/04; C07D 211/72

(52) U.S. Cl. 544/322; 568/706; 562/493; 568/583; 546/304

(57) ABSTRACT

The invention relates to the nitration of aromatic or heteroaromatic compounds, wherein an activated aromatic or heteroaromatic compound and a nitration agent, optionally in the presence of a solvent, are mixed intensely in a microreactor, and wherein the proportion of the nitration agent to the activated aromatic or heteroaromatic compound, the concentration of nitration agent in the reaction mixture, and the temperature are selected at levels such high that the nitration begins autocatalytically, and wherein the nitration product is obtained after leaving the microreactor and, optionally after an after-reaction time outside the microreactor.

The invention relates to the autocatalytic nitration of activated aromatic or heteroaromatic compounds in microreactors.

Usually nitrations of organic compounds are carried out with nitric acid or nitrating acid. Nitrating acid is a common name for mixtures of undefined stoichiometric composition of nitric acid and concentrated sulfuric acid, resp. derivatives and/or salts thereof. The nitration agent comprises at least one nitrogen compound which is able to release the electrophilic nitryl cation (NO₂)⁺ which is deemed to be the true nitration agent (comp. Nitration, Methods and Mechanisms, Series: Organic Nitro Chemistry Series, Olah, G. A., Malhotra, R., Narang, S. C., Verlag VCH, Weinheim 1989). After the nitration the nitration agent used has to be recycled more or less elaborately. The workup of nitration mixtures without amounts of sulfuric acid resp. of derivatives and/or salts thereof is easier than the workup of nitrating acids. Aromatic and hetero aromatic compounds often can be easily nitrated and often it is difficult to obtain selective introduction of only one nitro group.

Aromatic and heteroaromatic compounds can be distinguished in activated and deactivated compounds regarding their affinity to undergo nitrating reactions. Carbonyl, carboxyl or carboxyl ester groups have a deactivating effect. Hydroxy or alkoxy groups have an activating effect on reactivity.

Deactivated aromatic and heteroaromatic compounds are for example benzene, toluene, ethylbenzene, benzoic acid, phthalic acids or pyridine (comp. Olah, G. A. and Molnar, Á. Hydrocarbon Chemistry, Wiley & Sons, 1995, 419-421). Unsubstituted compounds like benzene and naphthalene show relatively less reactivity and hereinbelow are regarded to be deactivated compounds. Deactivated compounds preferably are nitrated with nitrating acid and often don't react at all or only slowly react with a nitration agent at low yields in the absence of sulfuric acid resp. derivatives and/or salts thereof.

In the meaning of the present invention and hereinbelow activated aromatic and heteroaromatic compounds are understood to be compounds having at least one hydroxy group and/or C₁₋₈-alkoxy group directly bound to an aromatic or heteroaromatic ring, such as for example phenol, p- and o-cresole, anisole, salicylic acid, 1- and 2-naphthalene, hydrochinone and 2-, 3- and 4-hydroxypyridine. Activating substituents decrease the activation energy for an electrophilic reaction (electrophilic attack) at the aromatic or heteroaromatic ring such that said ring can be nitrated by a nitration agent even in the absence of sulfuric acid resp. derivatives and/or salts thereof. Compounds having activating and deactivating substituents wherein the activating feature dominates and which therefore can be subject to an autocatalytic reaction when carried out in batch processes are also understood to be activated compounds. Salicylic acid and derivatives thereof, resp. ester and amides thereof are such, summarized, activated mixed compounds.

Nitrations of activated aromatic compounds in batch and semi batch processes are highly exothermic reactions and tent to “runaway” while large amounts of polymeric and/or poly-nitrated by-products are formed which have a detrimental effect on the product quality. The “runaway” is affected after the start of an autocatalytic nitration and leads normally to uncontrollable conditions with exponentially rise of released reaction enthalpy.

In the last years reactions in microreactors increasingly gained importance and are subject to numerous publications. Meanwhile, many companies offer microreactors in various models. For the purpose of cooling, for example of cooling in case of exothermic reactions, some microreactors are equipped with temperature adjustment channels within the microreactor body which can be perfused with a temperature adjustment media. A schematic graph of microreactors with active temperature adjustment can be found for example in Jähnisch, K. et al. Angew. Chem. 116, 2004, 410-451. Since mixing and reaction mechanisms in microreaction volumes are not fully understood yet neither choice of a suitable microreactor nor correct determination of reactions parameters is trivial.

Nitration of deactivated aromatic compounds in microreactors is known from DE-A-19935692 and WO-A-99/22858. In the known processes predominately nitrated are toluene or carbonylated compounds, wherein for nitration high activation energies have to overcome. An interruption of heat supply can rapidly cause a standstill of the reaction. Therefore, such reactions are easy to control in microreactors.

The method for nitration of deactivated aromatic compounds as disclosed in DE-A-19935692 and cannot be transferred for the purpose of nitration of activated aromatic and heteroaromatic compounds since the disclosed reactions follow an acid catalyzed electrophilic instead of an autocatalytic mechanism.

The investigations of Antes, J. et al. in Trans IChemE 81, 2003, 760-765 discloses the occurrence of concentration and temperature fluctuations in nitrations in microreactors. Large temperature fluctuations in addition to unwanted by-product formation enhance material fatigue of the microreactor. The danger that a microreactor suffers a crack limits assembling of larger microreactor units since leakage of nitration agents is an enormous security risk of highly environmental threatening potential.

The problem to be solved was to provide a process which allows continuous nitration of activated aromatic and heteroaromatic compounds in microreactors in steady and safe operation while sudden temperature and concentration fluctuations are maximal avoided. Furthermore, the formation of poly-nitrated reaction products and of polymeric by-products should be reduced.

This problem has been solved according to claim 1.

Claimed is a process for the nitration of aromatic or heteroaromatic compounds, wherein an activated aromatic or heteroaromatic compound and a nitration agent, optionally in the presence of a solvent, are intensely mixed in a microreactor, and wherein the proportion of the nitration agent to the activated aromatic or heteroaromatic compound and the concentration of nitration agent in the reaction mixture, and the temperature are selected at such high levels that the nitration starts autocatalytically in the microreactor, and wherein the nitration product is obtained after leaving the microreactor, optionally after an after-reaction time outside the microreactor.

In contrast to empirical knowledge in batch processes, wherein the onset of autocatalysis during nitrations of activated aromatic and heteroaromatic compounds leads to uncontrollable reaction conditions, it could be demonstrated that in a microreactor under continuous autocatalytic conditions the desired mono nitrated compounds could be obtained in good yields and purity. Furthermore, it could be shown that in a microreactor such permanent autocatalytic conditions can be well controlled.

In the inventive process a continuous operation with high material throughput, good yields and a good control of the reaction products can be reached. The reaction products obtained comprise remarkably little polymeric by-products and poly-nitrated compounds than such obtained from comparable batch processes.

The feed rate of the reaction partners, as well as amount and concentration of the nitration agent and the starting compound has to be set in a manner that autocatalytically nitration starts while contacting the reaction partners (at the beginning of the reaction volume or in a special designed mixing zone) and is maintained throughout the whole operation. The continuous reaction under autocatalytic conditions inhibits formation of so called “hot spots” within the microreactor. The size of the reaction volume has to be chosen in a manner to complete the nitration during the reaction time in the microreactor to a large extent. To prevent formation of interfering by-products batch wise after reaction times outside the microreactor should be kept as short as possible, preferable should be prevented at all.

To start the autocatalytic nitration of an activated aromatic or heteroaromatic compound in the inventive process at a defined temperature of the microreactor the proportion of the nitration agent to the activated aromatic or heteroaromatic compound and the concentration of nitration agent in the reaction mixture has to be present at least at a threshold value, preferably exceeds it, said level is defined in that below such level the autocatalysis comes to a standstill. Said defined temperature of the microreactor is the temperature of the temperature adjustment media used for temperature adjustment of the microreactor.

The start of the autocatalytic nitration is connected with a steep rise of released reaction enthalpy. When the start of the autocatalysis in the microreactor takes place within a couple of seconds, but still within the reaction volume, a steep rise of reaction heat flow can be observed, which stabilizes itself under continuous autocatalytic reaction conditions at a higher level. Such behavior can be only observed if stoichiometry and concentration of the reaction partners at a defined temperature are sufficient to just match the threshold value. If the autocatalysis is only caused locally by concentration variations, for example due to mixing effects and/or turbulent flow and starts and stops with uncontrollable behavior then so called “hot spots” are generated which come along with increased formation of polymeric by-products. Furthermore, then the microreactor is exposed to strong thermal stresses.

The rise of heat cannot be observed anymore if according to the inventive process the threshold value is permanently exceeded and autocatalysis immediately starts after mixing the reaction partners and is maintained within the microreactor until the end of the reaction. Exceeding the threshold value can be reached in principle and easy using a large excess of the nitration agent and/or at high temperatures. Then, autocatalysis certainly starts and a unitary product can be obtained. Optimization of the process regarding stoichiometry and concentration of the reaction partners can then be easily reached.

The threshold value has to be determined for any and each starting compound to be nitrated, each reactor type and also when the overall reaction conditions are changed. It is dependent of the starting compound, the nitration agent, the temperature, the concentration and amount of the reaction partners and therefore specifically for a defined process. Furthermore, in the presence of at least one C₂₋₅-carboxylic acid or an anhydride thereof the threshold value can be decreased. Close to the threshold value even little concentration variations within the microreactor, for example pumping system impacts, can cause a change between start and stop of the autocatalytic nitration.

Determination of the required amount of nitration agent per time interval, also regarding stoichiometry and concentration of the reaction partners, i.e. of the threshold value at defined reaction conditions, can be carried out easily by measuring the heat tone of the cooling media at its orifices of the microreactor. After start of the autocatalysis and under autocatalytic conditions the generation of blowholes can be observed.

The obtained reaction product, optionally after an after-reaction time in a further after-reaction volume, can be isolated or directly further reacted. The latter is suitably when subsequently used reaction partners and solvents are inert towards nitric acid.

Because in the inventive process neither sulfuric acid nor derivatives and/or salts thereof are required, after a phase separation only nitric acid resp. nitrous acid needs to be recycled, which renders the process simple and advantageously in view of commercial and environmental aspects.

Herein and hereinbelow the term “C₁₋ₙ-alkyl” is understood to be a straight or branched alkyl group of 1 to n carbon atoms. C₁₋₁₀-alkyl for example means methyl, ethyl, propyl, isopropyl, butyl, isobutyl, sec-butyl, tert-butyl, pentyl, 1,4-dimethyl-pentyl, hexyl, heptyl, octyl, nonanyl or decyl.

Herein and hereinbelow the term “C₁₋ₙ-alkoxy” is understood to be a straight or branched alkoxy group of 1 to n carbon atoms. C₁₋₁₀-alkyl for example means methoxy, ethoxy, propoxy, isopropoxy, butoxy, isobutoxy, sec-butoxy, tert-butoxy, pentyloxy, hexyloxy, heptyloxy, octyloxy, nonanyloxy or decyloxy.

Herein and hereinbelow the term “C₃₋ₙ-cycloalkyl” is understood to be a mono or bicyclic alkyl group of 3 to n carbon atoms. C₃₋₁₀-cycloalkyl for example means cyclopropyl, cyclobutyl, cyclopentyl, cyclohexyl, norbornyl, or cyclodecyl.

In the inventive process halogen is understood to be fluorine, chlorine, bromine and iodine.

Herein and hereinbelow the term “C₂₋₅-carboxylic acid” is understood to be an acid selected from the group consisting of acetic acid, propionic acid, butyric acid, isobutyric acid, and pentane acids. The aforementioned acids can be used as such as well as partly or completely halogenated derivatives thereof The definition of the anhydrides of “C₂₋₅-carboxylic acids” encompasses non-, partly or completely halogenated derivatives thereof, respectively, such as acetic acid anhydride or trifluoro acetic anhydride. Acids and anhydrides can be used as single compounds as well as mixtures thereof.

Herein and hereinbelow the term “C₁₋₃-alcohol” is understood to be an alcohol selected from the group consisting of methanol, ethanol, propanol or isopropyl alcohol.

In a preferred embodiment the activated aromatic or heteroaromatic compound comprises at least one substituent attached to the aromatic skeleton selected from the group consisting of hydroxy and C₁₋₆-alkoxy.

In a particularly preferred embodiment the activated aromatic or heteroaromatic compound is a mono or bicyclic compound.

Heteroaromatic compounds in the meaning of the inventive process preferably comprise one or two nitrogen atoms, like hydroxy pyridine or pyrimidine-4-ol. A bicyclic compound may also be partly hydrogenated and/or carry further substituents. An example for such partly hydrogenated bicyclic heteroaromatic compound is 5-hydroxy-1,2,3,4-tetrahydro-isochinoline.

Particularly preferred in the inventive process the activated aromatic or heteroaromatic compounds selected from the group consisting of phenols, salicylic acid and their derivatives, C₁₋₆-alkoxybenzenes, naphthalenes, C₁₋₆-alkoxynapht in the following halenes, hydrochinones, hydroxypyridines and hydroxypyrimidines, wherein each and any compound of the aforementioned compound classes optionally carry one or more additional substituents selected from the group consisting of halogen, C₁₋₁₀-alkyl, C₁₋₁₀-alkoxy and C₃₋₁₀-cycloalkyl.

The term salicylic acid means compounds, optionally derivatized at the carboxylic or hydroxy group or which carry further substituents attached to the ring selected from the group consisting of halogen, C₁₋₁₀-alkyl and C₁₋₁₀-alkoxy. C₁₋₁₀-alkyl group derivatized carboxylic groups are the respective salicylates, with C₁₋₁₀-alkoxy derivatized hydroxy groups are the respective acylsalicylic acids, for example acetylsalicylic acid.

In a particularly preferred embodiment the activated aromatic or heteroaromatic compound is selected from the group consisting of phenol, p- and o-cresole, anisole, naphthalene, hydrochinone, 2-, 3- and 4-hydroxypyridine, salicylic acid and acetylsalicylic acid.

The nitration agent for nitrating of activated aromatic or heteroaromatic compounds comprises at least one compound selected from the group consisting of diluted nitric acid, smoking nitric acid, and mixtures of nitric acid with C₂₋₅-carboxylic acids and/or anhydrides thereof, optionally in the presence of nitrogen dioxide, dinitrogen pentoxide and/or other nitrogen oxides.

Usually, nitrogen oxides are in balance with different species such as for example N₂O₄ [symbol] 2 NO₂.

Diluted nitric acid in the meaning of the inventive process is understood to be a mixture of HNO₃ with water able to nitrate, for example nitric acid (65%).

In a preferred embodiment the nitration agent comprises nitric acid (65%) and N₂O₂.

In a further preferred embodiment the C₂₋₅-carboxylic acid or the anhydride thereof is acetic acid or acetic acid anhydride.

In a further preferred embodiment the nitration agent comprises nitric acid (65%) and acetic acid and/or acetic acid anhydride.

In a preferred embodiment the stoichiometric proportion of the nitration agent to the activated aromatic or heteroaromatic compound is adjusted in a range of 1:1 to 4:1.

In a further preferred embodiment the volumetric proportion of the supplied nitration agent to the also solved activated aromatic or heteroaromatic compound is adjusted in a range of 1:5 to 1:1.

Preferably the microreactor is equipped with efficient temperature adjustment means, because after set in of the autocatalytic reaction the reaction n heat has to be discharged quickly. The microreactors used in the inventive process comprise at least two channels for separate supply of the reaction volume with the fluid phases of the starting compound and the nitration agent, optionally a mixing volume in front of the true reaction volume, a reaction volume wherein both phases are intensively mixed, at least one channel to guide the effluence out of the reaction volume and at least one temperature adjustment channel, which can be perfused by a fluid which can be temperature adjusted (temperature adjustment media).

The ability to control the temperature mainly is determined by the effective surface A and the heat transmission coefficient U. The effective surface A is defined of the theoretically ratio of the contact surface of the temperature adjustment channels to the reaction volume of the microreactor assuming that they would be directly adjoined and the heat could be exchanged without loss. The greater A, the more heat can be exchanged between the reaction volume and the temperature adjustment media. The heat transfer coefficient U specifies the heat flow in Watt, which is exchanged trough a 1 square meter joint surface at a temperature difference of 1 Kelvin between inner and outer surface. The greater U, the more heat can be exchanged between the reaction volume and the temperature adjustment media. Suitable microreactors for the inventive process can be obtained for example from the companies Corning Inc., N.Y., U.S.A., Ehrfeld Mikrotechnik GmbH, Wendelsheim, Germany or Cellular Process Chemistry Systems GmbH, Mainz, Germany.

In a particularly preferred embodiment the proportion of the effective surface (A) of the microreactor to its reaction volume is greater than 1000 m²/m³. The heat transmission coefficient (U) of the microreactor is preferably greater than 250 W/m³·K.

In a further preferred embodiment the effective surface A is greater than 2000 m²/m³ and the heat transmission coefficient of the microreactor is preferably greater than 500 W/m³·K. Presently, microreactors are offered having an effective surface A up to above 10000 m²/m³. It can be expected that in the future the figures A and U will further increase for microreactors.

Suitable microreactors for the present process can consist for example of a silicate glass, corrosion-resistant stainless steel or metal alloys or other corrosion-resistant vitreous, ceramic or metal compounds. Under corrosion-resistance is preferably understood the corrosion-resistance in the presence of a nitration agent, optionally under pressure and at elevated temperature.

In a preferred embodiment to total flow rate of all reaction partners per reaction volume (reaction flow) ranges from 1 to 100 g/min, particularly preferred from 5 to 50 g/min. For the feed of nitration agent to the optionally dissolved activated aromatic or heteroaromatic compound usually pumps are used. It is possible to use syringe pumps having a defined reservoir or hose, gyro, gear or rotary piston pumps.

In a preferred embodiment the residence time in the reaction volume is less than 30 seconds, preferably 20 seconds or less, particularly preferred 10 seconds or less.

In a preferred embodiment the nitration is carried out in the absence of a solvent.

Provided that the starting compounds at the reaction temperature can be pumped through the microreactor with or without the addition of water a solvent can be waived. As solvent inorganic and organic solvents can be used which do not react with the nitration agent, the starting compound and/or the reaction product. Particular appropriate is water and C₁₋₃-alcohols.

If, in case of less activated aromatics, for example of anisole, the residence time within the microreactor is not sufficient to reach complete conversion, an after reaction can take place in the effluent reaction mixture. Preferably such after reaction should not take place in a batch volume but in a continuously operated after reaction volume, which preferably can be temperature adjusted.

Said after reaction volume can be for example a commercially available retention module which can be temperature adjusted and which does not need to have any internal microstructures. Said after-reaction can be prevented by phase separation or diluting, for example by addition of water or C₁₋₃-alcohols. A moderate after-reaction normally has no detrimental influence on the product profile.

Depending on the composition and concentration of the reaction partners the reaction mixture in the reaction volume optionally has to be brought to an elevated temperature. That can be performed for example in that the flow of the temperature adjustment media through the temperature adjustments channels is very high and in that the reservoir of the temperature adjustment media is construed large enough. Additional factors which increase the heat exchange are for example a high heat capacity of the temperature adjustment media.

Preferably the reservoir of the temperature adjustment media in view of the microreactor volume is chosen such great that the microreactor can be regarded as being isothermal. Furthermore advantageously, the flow of the temperature adjustment media (temperature flow) through the microreactor is significantly larger than the flow of the reaction media (reaction flow). In a preferred embodiment the proportion of the reaction flow to the temperature flow ranges from 1:5 up to 1:20, preferably from 1:10 up to 10:20.

As temperature adjusting media preferably is selected a fluid having a high heat transmission ability. Such temperature adjustment media preferably comprise water, C₁₋₃-alcohols, glycerole and/or silicon oils, as well as mixtures thereof. Among others suitable are commercial available temperature adjustment mixtures such as for example Thermal M® (JULABO Labortechnik GMBH, D-77960 Seelbach), Silicone oil Renggli M40 (Renggli AG, CH-6343 Rotkreuz, Switzerland) or Syltherm XLT (DOW Chemical Company, USA).

In a preferred embodiment the microreactor is perfused of a temperature adjustment media at a temperature of 0 to 80° C., particularly preferred of 10 to 60° C.

At the beginning of the reaction the temperature in the microreactor corresponds to the temperature if the temperature adjustment media flow temperature. The temperature in the inner parts of the microreactors can be measured not easily after the start of the reaction. Therefore, the temperature of the temperature adjustment media after its leave of the microreactor (return temperature) is defined to be the reaction temperature. The return temperature in every case is higher than the flow temperature after starting the reaction. Preferably for a stable running of the autocatalytic reaction, the temperature difference between flow and return temperature is kept as small as possible. In a preferred embodiment said temperature difference maximal is 15° C., particularly preferred it is less than 10° C.

The nitration of phenol according to the inventive process at temperatures between 0 to 80° C. regularly leads to 65 to 80% total yield of nitrophenol.)

The nitration of phenol in the inventive process can be regulated, dependent of the conditions, to reach a good repeatable para/ortho distribution of nitrophenol in the range from 0.7 to 1.2.

In contrast to batch or semi batch processes the reaction products of the inventive process contains less polymeric and less poly-nitrated by-products.

The invention is exemplified by the following non-limiting examples.

### EXAMPLES

The following examples have been carried out in microreactors which meet the requirements of the inventive process. The composition of the solutions and the supply rates of the components resp. the mixtures in g/min are mentioned in Examples 1 to 33. The nitration agent HNO₃ and optionally further additives on pump 1 and the optionally solved starting compound on pump 2 have been separately supplied with two pumps at the mentioned supply rates and have been mixed in the microreactor. The reaction time results consequently depending on the supply rate and the amount used. The reaction temperature is in tables 1 to 4 further parameters such as the reaction temperature, raw yields and product formation are mentioned. In all examples the reaction could be carried out until the end of the reaction and the desired product has been obtained.

Comparative examples could be carried out as batch processes because in the literature no examples for nitration of activated aromatic compounds are disclosed. Additional information such as amounts and temperature can be found in tables 1 o 4. The examples V8 to 28 in table 3 have been analyzed only in a qualitative manner.

### Examples 1 and 2

Corning glass microreactor (Corning Inc.)

Temperature adjustment fluid: Water, 200 ml/min

Mixture 1: HNO₃ 65% (161 g, 1.66 mol)

Mixture 2: Phenol (765 g, 3.31 mol), AcOH (199 g), water (2296 g)

Phenol supply: 3.68 to 3.73 g/min

AcOH supply: 0.96 to 0.97 g/min

Water supply: 12.07 to 13.46 g/min

HNO₃ supply: 2.61 to 4.39 g/min

### Examples 3 to 8

Corning glass microreactor (Corning Inc.)

Temperature adjustment fluid: Water, 200 ml/min

Mixture 1: HNO₃ 65% (1000 g, 10.32 mol)

Mixture 2: Phenol (900 g, 9.56 mol), water (100 g)

Phenol supply: 2.75 to 2.77 g/min

Water supply: 1.97 to 1.99 g/min

HNO₃ supply: 3.10 to 3.13 g/min

### Examples 9 to 11

Corning glass microreactor (Corning Inc.)

Temperature adjustment fluid: Water, 200 ml/min

Mixture 1: HNO₃ 65% (1000 g, 10.32 mol)

Mixture 2: Phenol (900 g, 9.56 mol), water (100 g)

Phenol supply: 2.758 to 2.760 g/min

Water supply: 1.669 to 2.554 g/min

HNO₃ supply: 2.531 to 4.173 g/min

### Example 12

Corning glass microreactor (Corning Inc.)

Temperature adjustment fluid: Water, 200 ml/min

Mixture 1: HNO₃ 65% (100 g, 1.032 mol), water (225 g)

Mixture 2: Phenol (180 g, 1.91 mol), water (20 g)

Phenol supply: 2.68 g/min

Water supply: 12.54 g/min

HNO₃ supply: 3.06 g/min

### Example 13

Corning glass microreactor (Corning Inc.)

Temperature adjustment fluid: Water, 200 ml/min

Mixture 1: HNO₃ 65% (100 g, 1.032 mol), water (117 g)

Mixture 2: Phenol (180 g, 1.91 mol), water (20 g)

Phenol supply: 2.68 g/min

Water supply: 7.84 g/min

HNO₃ supply: 3.22 g/min

### Examples 14 and 15

Metal microreactor Ehrfeld 50 Mikron (Ehrfeld Mikrotechnik GmbH)

Temperature adjustment fluid: Silicone oil Renggli M40/Huber Thermostat, 800 ml/min

Mixture 1: HNO₃ 65% (1000 g, 10.32 mol)

Mixture 2: Phenol 90% (900 g, 9.56 mol), water (100 g)

Phenol supply: 2.71 g/min

Water supply: 1.98 g/min

HNO₃ supply: 3.12 g/min

### Example 16

Metal microreactor Ehrfeld 50 Mikron (Ehrfeld Mikrotechnik GmbH)

Temperature adjustment fluid: Silicone oil Renggli M40/Huber Thermostat

Mixture 1: HNO₃ 65% (402 g, 4.15 mol)

Mixture 2: Phenol 90% (199 g, 2.12 mol), water (597 g), AcOH (52 g)

Phenol supply: 1.72 g/min

AcOH supply: 0.45 g/min

Water supply: 6.41 g/min

HNO₃ supply: 2.31 g/min

### Example 17

Metal microreactor Ehrfeld 50 Mikron (Ehrfeld Mikrotechnik GmbH)

Temperature adjustment fluid: Silicone oil Renggli M40/Huber Thermostat, 800 ml/min

Mixture 1: HNO₃ 65% (75 g, 1.19 mol), water (175 g)

Mixture 2: Phenol 90% (45 g, 0.48 mol), water (5 g)

Phenol supply: 2.00 g/min

Water supply: 5.68 g/min

HNO₃ supply: 2.34 g/min

### Example 18

Metal microreactor Ehrfeld 50 Mikron (Ehrfeld Mikrotechnik GmbH)

Temperature adjustment fluid: Silicone oil Renggli M40/Huber Thermostat, 800 ml/min

Mixture 1: HNO₃ 65% (125 g, 1.98 mol), water (125 g)

Mixture 2: Phenol 90% (45 g, 0.48 mol), water (5 g)

Phenol supply: 2.89 g/min

Water supply: 3.71 g/min

HNO₃ supply: 3.39 g/min

### Example 19

Metal microreactor Ehrfeld 50 Mikron (Ehrfeld Mikrotechnik GmbH)

Temperature adjustment fluid: Silicone oil Renggli M40/Huber Thermostat, 800 ml/min

Mixture 1: HNO₃ 65% (164 g, 2.60 mol), water (88 g)

Mixture 2: Phenol 90% (45 g, 0.48 mol), water (5 g)

Phenol supply: 3.44 g/min

Water supply: 2.55 g/min

HNO₃ supply: 4.03 g/min

### Example 20

Metal microreactor Ehrfeld 50 Mikron (Ehrfeld Mikrotechnik GmbH)

Temperature adjustment fluid: Silicone oil Renggli M40/Huber Thermostat, 800 ml/min

Mixture 1: HNO₃ 65% (61 g, 0.96 mol), water (189 g)

Mixture 2: Phenol 90% (45 g, 0.48 mol), water (5 g)

Phenol supply: 1.50 g/min

Water supply: 6.48 g/min

HNO₃ supply: 2.02 g/min

### Example 21

Glass microreactor Corning (Corning Inc.)

Temperature adjustment fluid: Water, 200 ml/min

Mixture 1: HNO₃ 65% (1000 g, 10.32 mol)

Mixture 2: 1-Naphthalene (100 g, 694 mmol), AcOH (500 g)

1-Naphthalene supply: 1.49 g/min

AcOH supply: 7.44 g/min

Water supply: 0.68 g/min

HNO₃ supply: 1.26 g/min

### Examples 22 to 24

Glass microreactor Corning (Corning Inc.)

Temperature adjustment fluid: Water, 200 ml/min

Mixture 1: HNO₃ 65% (1000 g, 10.32 mol)

Mixture 2: p-Cresole (100 g, 925 mmol), AcOH (500 g)

p-Cresole supply: 1.12 to 1.13 g/min

AcOH supply: 5.62 to 5.67 g/min

Water supply: 0.34 to 0.70 g/min

HNO₃ supply: 0.64 to 1.30 g/min

### Examples 25 to 28

Glass microreactor Corning (Corning Inc.)

Temperature adjustment fluid: Water. 200 ml/min

Mixture 1: HNO₃ 65% (1000 g, 10.32 mol)

Mixture 2: Anisole (100 g, 925 mmol), AcOH (500 g)

Anisole supply: 0.56 to 1.13 g/min

AcOH supply: 2.82 to 5.67 g/min

Water supply: 0.36 to 0.71 g/min

HNO₃ supply: 0.66 to 1.31 g/min

### Examples 29 to 33

Glass microreactor Corning (Corning Inc.)

Temperature adjustment fluid: Water. 200 ml/min

Mixture 1: HNO₃ 65% (800 g, 8.25 mol)

Mixture 2: Salicylic acid (79 g, 570 mmol). AcOH (777 g)

Salicylic acid supply: 0.80 to 0.87 g/min

AcOH supply: 7.88 to 8.58 g/min

Water supply: 0.21 to 0.43 g/min

HNO₃ supply: 0.40 to 0.81 g/min

### Comparative Example V1

Phenol 90% (80 g, 0.77 mmol), AcOH (20.8 g) and water (250 ml) are mixed in a 1 L three neck vessel with heat jacket at 20° C. and intensively stirred. Within 30 min 65% nitric acid (112 ml, 1.53 mol) is dosed. After complete dosage of nitric acid the autocatalysis starts. A mixture of mono and poly-nitrated reaction products is formed which are difficult to separate. After addition of 100 ml CH₂Cl₂ and separation of the organic phase 20.7% nitrophenol is obtained having a para/ortho ratio of von 0.56.

### Comparative Examples V2 to V7

Comparative Examples V2 to V7 have been carried out analogously to Comparative Example V1 with exception of the amounts and temperatures given in Table 2. As can be seen from Table 3 in V3 to V7 the addition of AcOH could be avoided. Because of the formation of large amounts of polymeric by-products the content of hydrochinone, 2,4-dinitrophenol and 2,6-dinitrophenol partially could not analyzed.

### Comparative Example V8

1-Naphthalene (10 g, 69 mmol) and Acetic acid (47.6 ml) have been mixed in a 100 ml three neck vessel with temperature jacket at 20° C. and intensively stirred. Within 30 min nitric acid (65%, 10.2 ml, 139 mmol) have been dosed. After complete dosage of nitric acid the autocatalysis starts. A mixture is formed which is barely to separate comprising mono and poly-nitrated reaction products.

### Comparative example V9

p-Cresole (10 g, 92 mmol), acetic acid (28.6 ml) and water (20 ml) have been mixed in a 100 ml three neck vessel with temperature jacket at 20° C. and intensively stirred. Within 30 min nitric acid (65%, 13.5 ml, 185 mmol) have been dosed. After complete dosage of nitric acid the autocatalysis starts. A mixture is formed which is barely to separate comprising mono and poly-nitrated reaction products.

### Comparative Examples V10 and V11

p-Cresole (10 g, 92 mmol) and acetic acid (47.6 ml) have been mixed in a 100 ml three neck vessel with temperature jacket at 20° C. and intensively stirred. Within 30 min nitric acid (65%, 6.8 or 13.5 ml, 93 or 185 mmol, respectively) have been dosed. After complete dosage of nitric acid the autocatalysis starts. A mixture is formed which is barely to separate comprising mono and poly-nitrated reaction products.

### Comparative Example V12

Anisole (10 g, 92 mmol), acetic acid (28.6 ml) and water (20 ml) have been mixed in a 100 ml three neck vessel with temperature jacket at 20° C. and intensively stirred. Within 30 min nitric acid (65%, 13.5 ml, 185 mmol) have been dosed. After complete dosage of nitric acid the autocatalysis starts. A mixture is formed which is barely to separate comprising mono and poly-nitrated reaction products.

### Comparative Example V13

Anisole (10 g, 92 mmol) and acetic acid (47.6 ml) have been mixed in a 100 ml three neck vessel with temperature jacket at 20° C. and intensively stirred. Within 30 min nitric acid (65%, 13.5 ml, 185 mmol) have been dosed. After complete dosage of nitric acid the autocatalysis starts. A mixture is formed which is barely to separate comprising mono and poly-nitrated reaction products.

### Comparative Example V14

Salicylic acid (5.0 g, 36 mmol) and acetic acid (41.9 ml) have been mixed in a 100 ml three neck vessel with temperature jacket at 20° C. and intensively stirred. Within 30 min nitric acid (65%, 7.1 ml, 73 mmol) have been dosed. After complete dosage of nitric acid the autocatalysis starts. After the reaction has stopped the reaction mixture has been poured onto ice water (156 ml). The raw product is filtered off and washed with water. Yield: 3.57 g (19.5 mmol, 54.1%) Nitrosalicylic acid, comprising 5-nitrosalicylic acid (2.33 g, 35.3%) and 3-nitrosalicylic acid (1.24 g, 18.8%).

### Comparative example V15

Salicylic acid (6.8 g, 49 mmol) and acetic acid (63 ml) have been mixed in a 100 ml three neck vessel with temperature jacket at 75° C. and intensively stirred. Within 30 min nitric acid (65%, 7.1 ml, 73 mmol) have been dosed. After complete dosage of nitric acid the autocatalysis starts. After the reaction has stopped the reaction mixture is poured onto ice water (348 ml). The raw product is filtered off and washed with water. Yield: 4.7 g (26 mmol, 52.6%) nitrosalicylic acid, comprising 5-nitrosalicylic acid (3.12 g, 34.8%) and 3-nitrosalicylic acid (1.24 g, 18.8%).

TABLE 1
Examples 1 to 20

| Example | T | Phenol | | | p/o- | H-Chinone | 2,4-DNP | 2,6-DNP | P-PS |
|---|---|---|---|---|---|---|---|---|---|
| No. | [° C.] | [wt %] | HNO₃/Ed | Yield | Ratio | [%] | [%] | [%] | [%] |
| 1 | 60 | 15.9 | 1.59 | 75.5 | 1.01 | 2.4 | 0.8 | 0.0 | 8.6 |
| 2 | 45 | 15.9 | 1.77 | 74.8 | 1.05 | 1.6 | 0.0 | 0.6 | 9.0 |
| 3 | 5 | 58.0 | 1.69 | 74.1 | 0.93 | 2.9 | 3.2 | 0.0 | 6.5 |
| 4 | 15 | 58.0 | 1.68 | 70.0 | 0.93 | 2.9 | 4.2 | 1.3 | 7.2 |
| 5 | 25 | 58.0 | 1.69 | 70.0 | 0.92 | 3.1 | 5.5 | 1.9 | 6.9 |
| 6 | 35 | 58.0 | 1.69 | 68.5 | 0.94 | 3.2 | 6.6 | 2.3 | 7.1 |
| 7 | 45 | 58.0 | 1.70 | 64.7 | 0.94 | 2.8 | 7.4 | 2.4 | 9.1 |
| 8 | 55 | 58.0 | 1.69 | 64.9 | 0.94 | 3.1 | 8.7 | 2.8 | 8.9 |
| 9 | 20 | 58.0 | 1.37 | 77.1 | 0.98 | 3.8 | 4.2 | 1.9 | 7.4 |
| 10 | 20 | 58.0 | 1.98 | 68.7 | 0.96 | 2.3 | 6.8 | 2.7 | 7.0 |
| 11 | 20 | 58.0 | 2.26 | 64.7 | 0.93 | 2.0 | 7.8 | 3.0 | 7.8 |
| 12 | 55 | 17.6 | 1.70 | 74.8 | 0.94 | 0.9 | 0.0 | 0.0 | 8.6 |
| 13 | 55 | 25.5 | 1.80 | 75.6 | 0.92 | 1.4 | 1.1 | 0.0 | 1.6 |
| 14 | 10 | 58.0 | 1.72 | 71.2 | 0.91 | 0.8 | 5.9 | 0.8 | 6.4 |
| 15 | 25 | 58.0 | 1.73 | 67.9 | 0.95 | 0.8 | 8.0 | 2.3 | 7.4 |
| 16 | 45 | 20.1 | 2.00 | 75.3 | 0.72 | 0.6 | 0.4 | 0.0 | 11.4 |
| 17 | 55 | 26.0 | 1.75 | 75.0 | 0.81 | 1.1 | 0.1 | 0.0 | 6.5 |
| 18 | 55 | 43.8 | 1.75 | 74.4 | 0.89 | 0.6 | 2.5 | 1.7 | 4.5 |
| 19 | 55 | 57.4 | 1.75 | 70.2 | 0.91 | 1.8 | 4.5 | 2.2 | 4.4 |
| 20 | 55 | 18.8 | 2.00 | 68.3 | 0.74 | 0.6 | 0.0 | 0.0 | 19.6 |

TABLE 2
Examples V1 to V7

| Example | T | Phenol | | AcOH | | p/o- | H-Chinone | 2,4-DNP | 2,6-DNP | P-PS |
|---|---|---|---|---|---|---|---|---|---|---|
| No. | [° C.] | [wt %] | HNO₃/Ed | [g] | Yield | Ratio | [%] | [%] | [%] | [%] |
| V1 | 25-80 | 23.5 | 1.97 | 20.8 | 54.5 | 1.19 | 0.0 | 0.0 | 1.9 | 32.2 |
| V2 | 25-80 | 23.5 | 1.48 | 20.8 | 53.7 | 1.23 | 0.6 | 0.5 | 0.0 | 32.3 |
| V3 | 20-72 | 20.9 | 2.00 | 0.0 | 20.5 | 0.51 | n.a. | n.a. | n.a. | 79.5 |
| V4 | 20-72 | 20.9 | 2.00 | 0.0 | 20.7 | 0.56 | n.a. | n.a. | n.a. | 77.2 |
| V5 | 10-66 | 20.9 | 2.00 | 0.0 | 31.9 | 0.67 | n.a. | n.a. | n.a. | 72.1 |
| V6 | 0-55 | 20.9 | 2.00 | 0.0 | 29.7 | 1.15 | n.a. | n.a. | n.a. | 64.7 |
| V7 | −10-8 | 20.9 | 2.00 | 0.0 | 47.1 | 1.69 | n.a. | n.a. | n.a. | 64.3 |

TABLE 3
Examples V8 to 28

| | | | | Starting | | | | Formation of |
|---|---|---|---|---|---|---|---|---|
| Example | T | Starting | HNO₃/Ed | compound | AcOH | Water | rel. NMR | polymeric by- |
| No. | [° C.] | compound | [mol/mol] | [g] | [g] | [g] | purity | products |
| V8 | 17-33 | 1-Naphthalene | 2.0 | 10.0 | 50.0 | 0.0 | + | +++ |
| 21 | 38 | 1-Naphthalene | 1.83 | 10.0 | 50.0 | 0.0 | +++ | + |
| V9 | 17-25 | p-Cresole | 2.0 | 10.0 | 30.0 | 20.0 | + | +++ |
| V10 | 17-25 | p-Cresole | 2.0 | 10.0 | 50.0 | 0.0 | + | +++ |
| V11 | 17-33 | p-Cresole | 2.0 | 10.0 | 50.0 | 0.0 | + | +++ |
| 22 | 29 | p-Cresole | 0.97 | 10.0 | 50.0 | 0.0 | +++ | + |
| 23 | 29 | p-Cresole | 1.99 | 10.0 | 50.0 | 0.0 | +++ | + |
| 24 | 47 | p-Cresole | 1.01 | 10.0 | 50.0 | 0.0 | +++ | + |
| V12 | 16-17 | Anisole | 2.0 | 10.0 | 30.0 | 20.0 | + | +++ |
| V13 | 16-19 | Anisole | 2.0 | 10.0 | 50.0 | 0.0 | + | +++ |
| 25 | 38 | Anisole | 1.94 | 10.0 | 50.0 | 0.0 | +++ | + |
| 26 | 55 | Anisole | 2.01 | 10.0 | 50.0 | 0.0 | +++ | + |
| 27 | 72 | Anisole | 1.99 | 10.0 | 50.0 | 0.0 | +++ | + |
| 28 | 72 | Anisole | 2.02 | 10.0 | 50.0 | 0.0 | +++ | + |

TABLE 4
Examples V14 to 33

| | | | | | p/o- | | |
|---|---|---|---|---|---|---|---|
| Example | T | SA | HNO₃/Ed | Yield | Ratio | 3-NSA | 5-NSA |
| No. | [° C.] | [wt %] | [mol/mol] | [%] | [—] | [%] | [%] |
| V14 | 45 | 15.9 | 1.10 | 54.7 | 1.9 | 35.3 | 18.8 |
| V15 | 75 | 15.9 | 1.50 | 71.5 | 2.0 | 34.8 | 17.8 |
| 29 | 75 | 15.9 | 1.0 | 69.5 | 1.7 | 44.1 | 25.4 |
| 30 | 75 | 15.9 | 1.25 | 81.0 | 1.7 | 51..0 | 33.0 |
| 31 | 75 | 58.0 | 1.50 | 89.5 | 1.6 | 55.7 | 33.8 |
| 32 | 75 | 58.0 | 1.75 | 89.4 | 1.7 | 56.4 | 33.0 |
| 33 | 75 | 58.0 | 2.00 | 86.9 | 1.7 | 54.2 | 32.7 |

### Definition of the Column Entries in Tables 1 to 4:

T=reaction temperature

Phenol=Phenol part by weight

Starting compound=Starting compound

HNO₃/Ed=molar partition of HNO₃ to Starting compound

Yield=Raw yield

p/o-Ratio=Partition of para- to ortho product

H-Chinone=Hydrochinone

2,4-DNP=2,4-Dinitrophenol

2,6-DNP=2,6-Dinitrophenol

P-SP=polymeric by-products

SA=Salicylic acic

3-NSA=3-Nitrosalicylic acid (para)

5-NSA=5-Nitrosalicylic acid (ortho)

Starting compound [g]=Amount of starting compound in the reaction mixture

Starting compound [wt %]=weight percent of starting compound in the reaction mixture

AcOH=Amount of acetic acid in the reaction mixture

Water=Amount of water in the reaction mixture

rel. purity NMR=qualitative interpretation of purity of products (+=bad, +++=very good) according to the ¹H-NMR Spectra

Formation of polymeric by-products=qualitative interpretation of thin layer chromatograms (+=few polymeric by-products, +++=many polymeric by-products)

1. A process for the nitration of an aromatic or heteroaromatic compound, having at least one hydroxyl group and/or C₁₋₈-alkoxy group directly bound to the aromatic or heteroaromatic ring, wherein the aromatic or heteroaromatic compound and a nitration agent, optionally in the presence of a solvent, are intensely mixed in a microreactor, and wherein the proportion of the nitration agent to the aromatic or heteroaromatic compound and the concentration of nitration agent in the reaction mixture are selected at such high levels that the nitration starts autocatalytically in the microreactor, and wherein the nitration product is obtained after leaving the microreactor, optionally after an after-reaction time outside the microreactor.

2. A process according to claim 1, wherein the alkoxy group is C₁₋₆.

3. A process according to claim 1, wherein the aromatic or heteroaromatic compound is a mono or bicyclic compound.

4. A process according to claim 1, wherein the aromatic or heteroaromatic compound selected from the group consisting of phenols, salicylic acid and their derivatives, C₁₋₆₋alkoxybenzenes, naphthalenes, C₁₋₆₋alkoxy-naphthalenes, hydrochinones, hydroxy-pyridines and hydroxypyrimidines.

5. A process according to claim 1, wherein the nitration agent comprises at least one compound selected from the group consisting of diluted nitric acid, smoking nitric acid, and mixtures of nitric acid with C₂₋₅₋carboxylic acids and/or anhydrides thereof, optionally in the presence of nitrogen dioxide, dinitrogen pentoxide and/or other nitrogen oxides.

6. A process according to claim 1, wherein the stoichiometric proportion of the nitration agent to the aromatic or heteroaromatic compound is adjusted in a range of 1:1 to 4:1.

7. A process according to claim 1, wherein the proportion of the effective surface (A) of the microreactor to its reaction volume is greater than 1000 m²/m³ and the heat transmission coefficient (U) of the microreactor is greater than 250 W/m³·K.

8. A process according to claim 1, wherein the residence time in the reaction volume is less than 30 seconds.

9. A process according to claim 1, wherein the nitration is carried out in the absence of a solvent.

10. A process according to claim 1, wherein the microreactor is perfused of a temperature adjustment media at a temperature of 0 to 80° C., particularly preferred of 10 to 60° C.
